(19) United States
(12) Patent Application Publication
(10) Pub. No.: US 2016/0278746 A1
(43) Pub. Date: Sep. 29, 2016

(54) REAL-TIME TRACKING FOR MRI-GUIDED BREAST BIOPSY

(71) Applicant: GENERAL ELECTRIC COMPANY, SCHENECTADY, NY (US)

(72) Inventors: Ileana Hancu, Niskayuna, NY (US); Robert David Darrow, Scotia, NY (US); Eric William Fiveland, Niskayuna, NY (US); Mauricio Castillo-Effen, Rexford, NY (US); Seung-Kyun Lee, Cohoes, NY (US)

(21) Appl. No.: 14/671,252

(22) Filed: Mar. 27, 2015

(51) Int. Cl. A61B 10/02; A61B 5/00; A61B 90/11; A61B 5/055

(52) U.S. Cl. CPC A61B 10/0266; A61B 5/055; A61B 5/4312; A61B 90/11

(57) ABSTRACT

The system and method of the invention pertains to an MR-guided breast biopsy procedure, specifically as to real-time tracking and navigation of a biopsy device. More particularly, the system utilizes a diagnostic imaging modality such as magnetic resonance imaging (MRI) to locate lesions in a human breast while utilizing an inertial measurement unit (IMU) to track advancement of a biopsy device in real-time. The invention simplifies the workflow of MRI-guided breast biopsies, shortens the time needed to perform the biopsy, decreases cost, and increases accuracy. This is achieved by enabling real-time visualization of the biopsy device as it advances towards the targeted lesion.

### STATEMENT AS TO FEDERALLY SPONSORED RESEARCH & DEVELOPMENT

This invention was made with Government support under contract number R01CA154433 awarded by the National Institutes of Health through the National Cancer Institute. The Government has certain rights in the invention.

### FIELD

Embodiments relate generally to the field of imaging and biopsy, and more particularly to real-time tracking for Magnetic Resonance Imaging (MRI) guided biopsy.

### BACKGROUND

While 60% of the sites owning a full body MRI scanner perform breast MRI exams, only 5% perform MRI-guided breast biopsies. A number of reasons explain why MRI-guided biopsies are not more common. To better understand the shortcomings of the procedure as used in the art, the tools of the procedure are highlighted in FIG. 1, and described as follows. The biopsy setup 100 is depicted in FIG. 1 as an assembled biopsy setup (a) and as separate components (b).

While a woman patient is positioned supine on a breast coil, the breast to be biopsied is compressed between a coarse plastic grid 101 and an immobilization, or compression plate (e.g. behind the grid in the lower-most image of FIG. 1). The grid typically has openings 103 sized 2 cm×2 cm. Each of the grid openings accepts a sub-grid insert 105 which contains a matrix of 3×3 insertion locations 107. The woman is advanced in the MRI scanner, and a contrast agent is administered to localize the lesion. A fiducial marker on the coarse grid 101 is used to identify lesion position relative to the biopsy device. The biopsy location is then defined by the clinician. This may be a time-consuming step, as the screening and biopsy images may be acquired in different orientations. Moreover, the screening images are acquired with the breasts uncompressed, while the biopsy images are acquired with the breast compressed. The compression can limit perfusion, hence causing the suspicious lesion not to enhance anymore.

Following lesion identification, software computes the entry position (i.e., coarse grid position and grid insert position) and lesion depth, and reports it on the computer screen in the scanner control room. Typically, given the single degree of freedom available for biopsy tool advancement, a single entry location is possible for a given lesion. At this point, the patient is removed from the magnet, while the compressed breast containing the lesion remains in a fixed position. The clinician enters the scanner room, identifies the entry location (i.e., coarse grid row and column, as well as grid insert row and column) and inserts a stylet 109 into an introducer 111, then into the grid insert 105, and then into the coarse grid 101. Once a particular grid entry point is chosen, a single degree of freedom is allowed for the biopsy device, which can only advance orthogonal, at right angles, to the grid plane. The introducer has depth markings, and a moveable, friction-fit ring 112 to control the depth of its insertion into the breast. The stylet is advanced to the approximately depth into the breast (defined manually by the setting of the friction-fit ring by the physician), then replaced with a plastic obturator 113. The medical team leaves the room and the patient is re-imaged to confirm if the tip of the obturator is at the location of the lesion. Assuming image confirmation, the patient is taken out of the magnet again, the obturator is replaced with a biopsy gun 108, and biopsy samples are taken (e.g., by rotating the biopsy gun multiple times). At the end of the procedure, the biopsy gun is replaced with the obturator, the patient is advanced to the scan position, and another image is acquired, for visual assessment of biopsy success.

Prior art techniques, such as that described above, make MRI-guided breast biopsy workflow cumbersome, resulting in a procedure completion time of 30-60 min. This utilizes a large fraction of MRI scanner time, numerous personnel (e.g., interventional radiologist, nurse and scanning technologist), and drives cost high. The MRI-guided biopsies are conducted without visualization of the lesion or direct guidance. The lesions can only be visualized for ˜10 minutes after the contrast agent was injected, while the woman is inside the MRI magnet. The biopsies are performed, however, outside the MRI magnet, with the women on the MRI table. Accuracy is limited given the 6 mm (or 8 mm) distance between possible adjacent insertion points (and depending on whether the adjacent insertion points fall within the same opening of the coarse grid or not). See FIG. 1. Thus, this also limits the locations where the tip of the biopsy needle can reach. Larger than needed tissue volume is therefore extracted to sample at least a fraction of the enhanced lesion.

Note that during the insertion of the surgical tools, such as the stylet or the biopsy gun, no imaging is performed. The surgical instruments (e.g. stylet, obturator, biopsy gun, etc.) are inserted blindly such that neither the instrument is visualized, nor any construct/image of the instrument.

In comparison, core biopsies, as typically performed for breast lesions under ultrasound guidance, employ 11-18 gauge needles (with 14 gauge being typical) and extract about 4 samples/lesion (for about 80 mg total mass of extracted tissue); vacuum assisted biopsies for MRI-guided biopsies typically employ 9 gauge needles and extract about 8 samples/lesion (for a total mass of extracted tissue of about 1.5 g). The lack of real-time guidance, the limited number of entry points, and the orthogonal advancement make it difficult for the clinician to access lesions requiring high accuracy, such as the ones close to silicone implants. In addition, lesions located outside of the compression grid (e.g., posterior) are very difficult to access with any kind of accuracy. Furthermore, large blood vessels cannot be avoided; accidental puncture can lead to the creation of a hematoma(s) and morbidity to the patient. In fact, about 1.5% of MRI-guided biopsies are interrupted due to excessive bleeding. Assessment of the biopsy procedure is done at the end visually, with no quantitative tool available to confirm the fraction of the lesion removed. Furthermore, by the end of the procedure, the contrast agent may have already washed out, providing different contrast and slightly different geometry that renders this visual assessment inaccurate.

Given the shortcomings described above, cancers can be missed. Follow-up MRI, after benign and imaging-histology concordant MRI-guided biopsies, has shown that 8-12% of targeted lesions were inadequately sampled; and malignancy was ultimately diagnosed in 14-18% of these cases. Follow-up after benign and imaging-histology discordant biopsies indicated malignancies in 13-44% of the lesions initially diagnosed as benign. False negative rates as high as about 12% have been reported for MRI-guided biopsies. This demonstrates the need for improved procedures.

While tracking of surgical instruments during interventional procedures has been implemented prior, a need exists to track with high precision, over a relatively large region, in a strong, inhomogenous background magnetic field. Optical tracking of part of an instrument outside of the body may be possible but proves difficult when the hand of a clinician is interposed between the instrument and the source of light or detector. More typically, instrument tracking is done using electromagnetic tracking. In practical implementations, in order to increase the tracking range, both transmitters and receivers of the electromagnetic tracking system have ferromagnetic cores, hence increasing their sensitivity. The presence of such ferromagnetic objects in an MRI suite creates a safety hazard, making the electromagnetic tracking systems not suitable to work in the high magnetic fields existent in the fringe field of the MRI machines, where breast biopsies are performed. Removal of the ferromagnetic cores can decrease the tracking range below what is needed, or increase the sensor dimensions above what is reasonable.

While MRI provides excellent soft-tissue contrast and the ability to distinguish tumor margins, it is clear that an improved system for MRI guided biopsy is needed to shorten the duration of the procedure and increase its accuracy. The tracking described in the invention will enable visualization of the surgical instrument as it advances to the lesion, thus improving the accuracy of the procedure. Reduction in the number of imaging steps to validate the position of the instrument will also reduce the duration of the procedure. The system will beneficially operate with imaging technology as used for a specific interventional procedure.

To fulfill the potential of breast MRI as the test with unparalleled sensitivity for breast cancer detection, a simple and accurate solution for MRI guided breast biopsies needs to be devised. Widespread acceptance and practice of these biopsies, as currently implemented, is not practical or economically feasible due to the time, expense and high level of skill associated with current workflow. Further, given the percentage of false negatives, inaccuracy is a significant concern. The lack of a simple solution for MRI-guided breast biopsies will ultimately stunt the growth of breast MRI as a screening modality, and will prevent many women from benefitting from this very sensitive test. A need exists to fundamentally simplify and increase the accuracy of MRI-guided breast biopsy procedures. The invention will address some shortcomings of present day MR-guided biopsy procedures, rendering the procedures shorter in duration, more accurate, and cheaper.

### SUMMARY

The system and method of the invention pertains to an MR-guided breast biopsy procedure, specifically as to MRI guided breast biopsies are expensive, difficult to perform, and inaccurate, requiring more tissue than needed to be extracted, in order to insure that the suspected lesions was sampled accurately. This invention simplifies the workflow of the MRI guided breast biopsies, shortens the time needed to perform these biopsies, decreases their cost, and increases their accuracy. This is achieved by enabling real-time visualization of the biopsy device as it advances towards the targeted lesion.

While MRI provides excellent soft-tissue contrast and the ability to distinguish tumor margins, it is clear that an improved system for tissue biopsy is needed to better target a desired lesion with a surgical instrument. Embodiments of the improved system include a real-time tracking and navigation technique which provides precise, continuous, virtual three-dimensional (3D) visualization of the surgical instrument as it advances to the target lesion during the procedure. The real-time tracking described in the invention keeps track of the position of the surgical instrument (e.g., dynamic positioning and movement) during the procedure, thus ensuring that the correct designated tissue is sampled, hence reducing the false negative rate of the procedure. The system beneficially operates with imaging technology as used for a specific interventional procedure.

One embodiment of the invention includes a system for real-time tracking and navigation during magnetic resonance imaging (MRI) guided intervention, the system comprising: a sensor combination attached to an interventional instrument, wherein the sensor combination includes at least one gyroscope and at least one accelerometer, with the sensor combination recording a plurality of sensor measurements; a computer processor executing an algorithm that relates the sensor measurements to states of the system, the states comprising position, velocity, acceleration, and angular velocity; and a display presenting a real-time, three-dimensional (3D) visualization of a fixed point on the interventional instrument overlayed on the anatomy under study, the designated anatomical target, wherein the sensor combination is an inertial measurement unit (IMU).

In one aspect, the sensor combination further comprises at least one magnetometer. In another aspect, the MRI guided intervention is performed in a fringe field of an MRI scanner. One embodiment utilizes an IMU that includes at least three accelerometers, three gyroscopes, and three magnetometers along three orthogonal axes. The system can further comprise a measured or simulated 3D map of a magnetic field at a location of the MRI guided intervention that is used by the algorithm to increase localization accuracy. In one embodiment, the fixed point on the interventional instrument is within a tip of a surgical instrument and is displayed virtually on the anatomy under study. The display depicts movement of the tip of the surgical instrument as it advances towards a target during the MRI guided interventional procedure.

Embodiments of the invention comprise one or more images of the anatomy under study acquired prior to the MRI guided intervention. The sensor combination may be attached to a stylet or any biopsy device. The sensor combination may include at least one MEMS device.

The invention has been implemented in MRI guided intervention, specifically in the field of biopsy, and more particularly as it relates to breast biopsy, where the target is a breast lesion. The IMU as utilized is located on the interventional instrument and the breast biopsy is performed next to a breast coil in the MRI room. The MR guided intervention is performed in a fringe field of an MR scanner to provide accurate real-time guidance. Thus, embodiments may implement an occupancy grid map correlated with the sensor measurements obtained from the sensor combination.

The system, in one aspect, comprises magnetometers which can are Hall-effect sensors providing the components of a magnetic field in a frame of reference of the instrument, while the gyroscopes and the accelerometers provide the orientation of the instrument.

The method of real-time tracking and navigation during magnetic resonance imaging (MRI) guided intervention includes providing a system for real-time tracking and navigation during magnetic resonance imaging (MRI) guided intervention, as described, advancing a medical device towards an anatomical target; tracking the medical device during the step of advancing, wherein the step of tracking comprises obtaining sensor measurements from the sensor combination and transforming the sensor measurements into a state of the medical device. The state represents position, velocity, and orientation, without external reference. The state may also represent one or more of a change in the position, a change in the velocity, and a change in the orientation, alone or in combination. A step of extending the algorithm models drift and bias of the IMU. Further, a step of correcting the algorithm to update a predicted state, by incorporating noise and map errors in a probability density function, accounts for occupancy grid map errors in navigation. A technique of simultaneous localization and mapping (SLAM) may be utilized to refine mapping of the at least one magnetic field in a biopsy region for each individual scanner.

Embodiments of the invention include a step of advancing a biopsy device through the biopsy grid to a target location of the lesion, with visualization in real-time during the biopsy procedure, such that the step of advancing the biopsy device proceeds without piercing any of the one or more blood vessels. In addition, the method may comprise a step of using computer-aided detection of the target. Detailed descriptions of various embodiments are described as follows.

### BRIEF DESCRIPTION OF THE DRAWINGS

FIG. 1 (PRIOR ART) is an illustration of the tools as currently utilized in biopsy: (a) the assembled biopsy setup; and (b) the separate components as utilized for biopsy procedures.

FIG. 2 provides MRI-guided biopsy workflow performed within (a) prior utilized thirty to sixty (30-60) minute timeframes, as compared to (b) an embodiment of the invention implementing real-time tracking during MRI guided biopsy workflow, performed within a timeframe of about fifteen (15) minutes or less.

FIG. 3 illustrates a schematic of an embodiment of the invention depicting the biopsy setup.

FIG. 4 illustrates a flow diagram in one embodiment that depicts the operation of real-time tracking implemented with an MRI guided interventional procedure.

FIG. 5 depicts an embodiment of the invention including a biopsy setup in an MRI scanner room, adjacent a breast coil.

FIG. 6 depicts an embodiment of the invention including magnetic field reported by a magnetometer and position reported by an optical encoder, demonstrating a direct relationship between magnetic field and position.

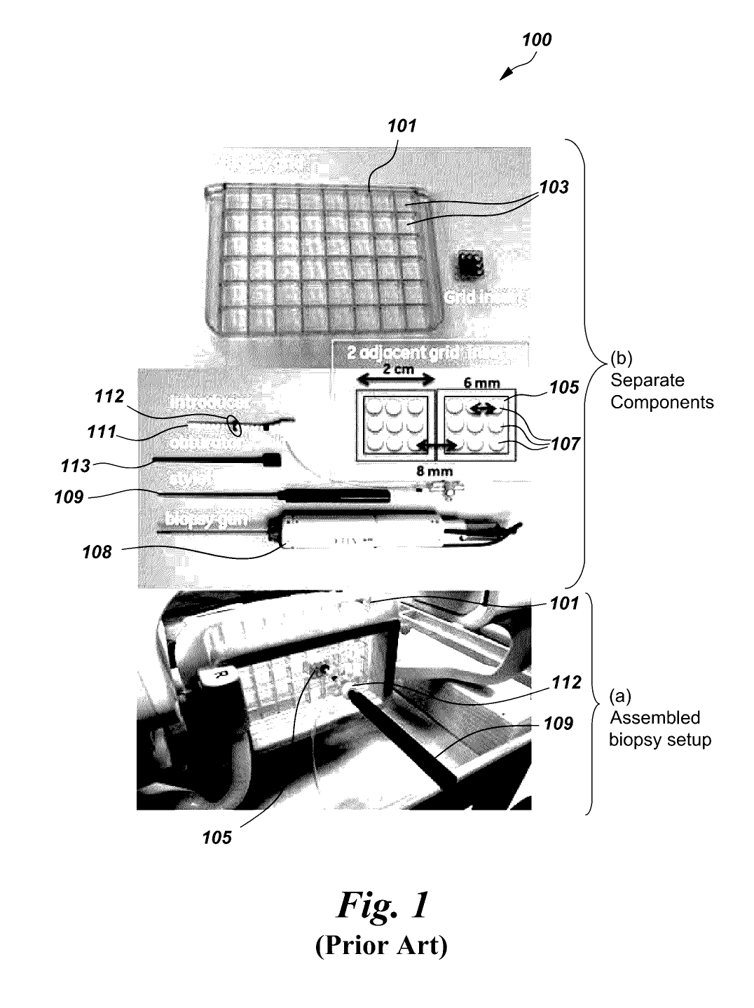

### DETAILED DESCRIPTION

Various embodiments will be better understood when read in conjunction with the appended drawings. It should be understood that the various embodiments are not limited to the arrangements and instrumentality shown in the drawings.

Embodiments of tracking technologies for the biopsy device have been designed to work in the strong, inhomogeneous fringe field of the MRI magnet. The desired tracking range is about 20 cm×20 cm×50 cm; tracking is intended to give positioning of the surgical instrument with an accuracy of better than about 2 mm. One tracking approach is based on a set of 3-axis Hall-effect-gyroscope-accelerometer sensors: respectively, sensors/transducers yield varying output voltages in response to the different magnetic fields sensed, as well as for different accelerations and angular velocities. Other motion processing technology may be utilized as well, and such sensors implemented in various arrangements and combinations.

Biopsies are performed in a region of inhomogeneous magnetic field where a unique relationship exists between the three components of the magnetic field and position. While the Hall-effect sensors can provide the three components of the magnetic field in the instrument's frame of reference, the gyroscope and accelerometer provide the instrument's orientation, hence enabling full position determination. This is the first time when an inertial measurement unit (IMU) is used for high accuracy tracking in a surgical setting, benefitting from the unique advantage offered by the strong, position-dependent magnetic field.

Real-time tracking of the biopsy tool of the invention utilizes IMU sensors, an inexpensive solution that does not clutter the room with additional hardware. Embodiments of the invention employ an IMU (e.g. similar, in principle, to that utilized in plane or missile tracking) for high accuracy position determination in a surgical application. This approach may also utilize high precision mapping of the magnetic field in the biopsy region for each individual scanner if the simulated maps do not correspond to the field measured in real life. A technique known as Simultaneous Localization and Mapping (SLAM) may also be used to refine mapping of the magnetic field in the biopsy region for each individual scanner, such as when the simulated maps do not correspond to the field measured in real life.

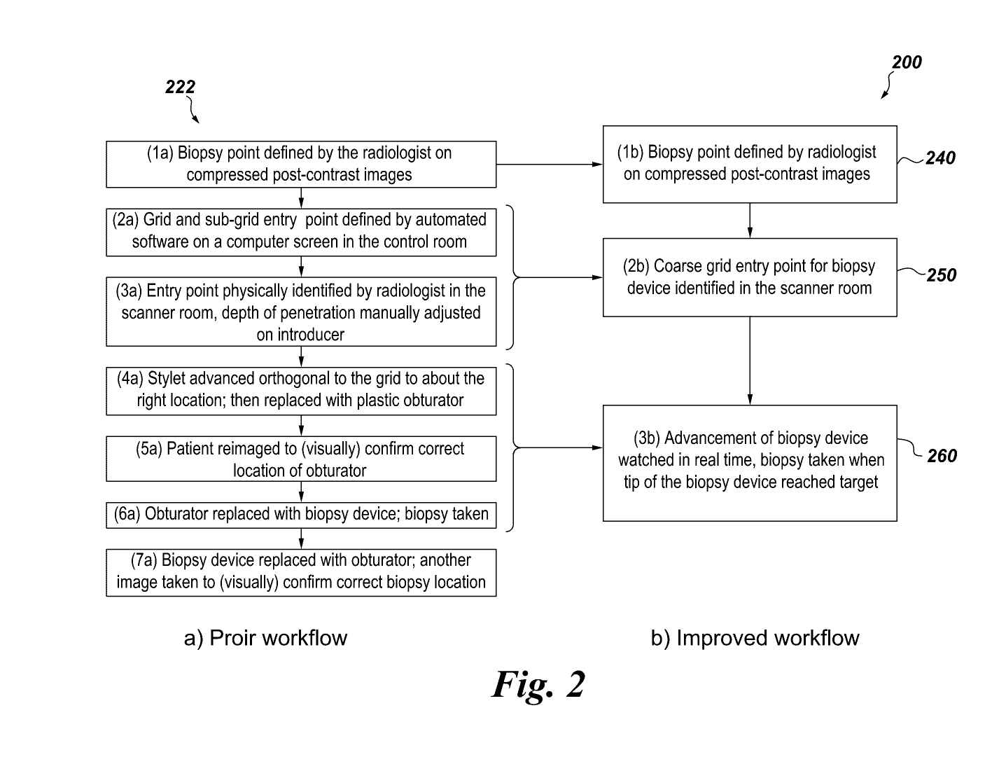

In one embodiment of the invention, the improved MR-guided breast biopsy procedure is reduced from prior 30-60 min procedures to duration of about 15 minutes or less, and with greater accuracy. FIG. 2(a) depicts the workflow 222 of a breast biopsy procedure as currently known in the art. Because the imaging is performed in the MRI magnet, and the biopsy performed outside the magnet (i.e. essentially blindly), a lot of back and forth steps are utilized to confirm by imaging that the biopsy procedure is performed at the designated location, i.e. where the lesion appeared on the images.

FIG. 2(b) depicts a schematic to compare workflow 200 in the present invention as utilized with technology of the invention, in contrast to the inefficiencies as listed in the prior workflow 222. In FIG. 2, the systems of 30-60 minute duration (e.g. FIG. 2(a)) include the following steps: (la) The radiologist identifies the biopsy location on the interventional (MRI) images (e.g. often on compressed post-contrast images); (2a) the grid and sub-grid entry point are defined by automated software on a computer screen in the control room; (3a) the entry point is then physically identified by the radiologist in the scanner room, with the depth of penetration manually adjusted on the introducer; (4a) the stylet is then advanced orthogonally through the grid to about the desired location, then replaced with the plastic obturator; (5a) the patient is re-imaged to confirm appropriate location of the tip of the obturator; (6a) the patient is removed from the scanner, the obturator is replaced with biopsy device and the biopsy then taken; and (7a) the biopsy device is replaced with the obturator, followed by the patient re-entering the magnet for re-imaging confirmation, e.g. another image then taken to visually confirm that the lesion was sampled at an appropriate biopsy location.

In one embodiment of the improved method, as shown in FIG. 2(b), workflow 200 illustrates a simplified procedure for biopsy. With real-time tracking of the surgical instrument, the prior back and forth steps are no longer needed. An embodiment of the method includes as follows: (1b) A patient is imaged to find a lesion, such that the radiologist defines a biopsy point on compressed post-contrast images (240); (2b) the patient is removed from the magnet and a coarse grid entry point is then identified, representing the entry point for a biopsy device (250); and (3b) the biopsy device is advanced to a target lesion, the device visualized in real-time, such that the biopsy is taken when the tip of the biopsy device reaches the target lesion and ensures that the biopsy is taken from the target lesion (260). This reduces procedure time and expense, while also facilitating more efficient patient care.

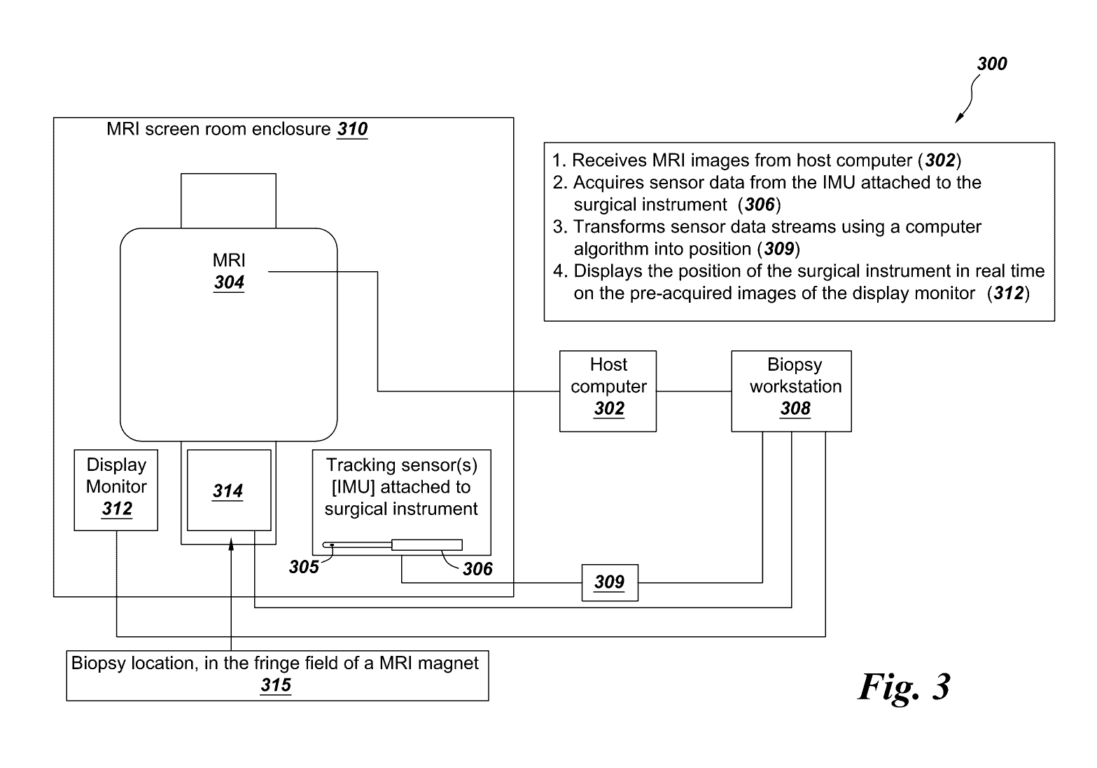

Embodiments of the invention provide the interventional radiologist with real-time visualization of his/her actions during a biopsy procedure. FIG. 3 presents an illustration of the physical changes in hardware implemented in the biopsy setup for the real-time tracking system 300, including connectivity and the role of each component. Specifically, FIG. 3 illustrates a system 300 where a computer 302 receives data in real-time from both an MRI scanner 304 and the tracking sensors 305 which are attached to the surgical instrument 306, both separate from the biopsy workstation 308 by way of the MRI screening room enclosure 310. Near the MRI scanner 304 are a display monitor 312 and a compression grid 314. The biopsy location is in the fringe field 315 of the MRI magnet 304. The biopsy workstation 308 receives MRI images from the host computer 302; acquires sensor data from the sensor combination, IMU 305, attached to the surgical instrument 306; transforms sensor data streams into position locators using a computer algorithm 309; and then registers, reformats, and sends three-dimensional (3D) images to the in-room display 312 for visualization. The sensor combination (IMU) is a mini-scale device attached at a distal or proximal end of the instrument. For exemplary purposes, the device is a box attached to the inside hollow tube body 311 of the instrument 306 (e.g. a stylet 306). The IMU may be positioned with the instrument to be tracked in various manners, including direct attachment of sensors to the body or tip of the instrument, internal or external to the tube body 311, or integral therewith.

In one aspect, the biopsy workstation sends desired information (e.g., real-time display of biopsy advancement) to the display monitor 312 located in the MRI screen room enclosure 310. As depicted, the biopsy workstation 308 and the host computer 302 include separate respective processors. In another aspect, the processor of the host computer 302 may be included in the biopsy workstation 308, or part of the host computer of the scanner can perform the steps as described.

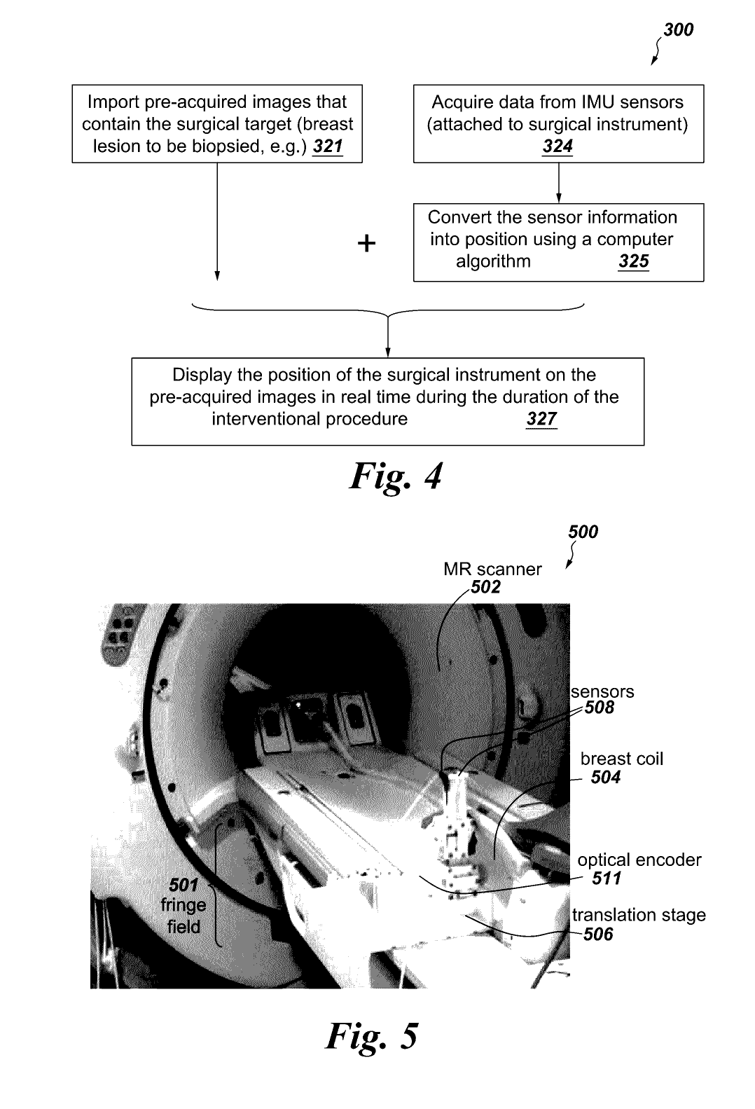

As depicted in FIG. 4, the actions of the computer processor 302, which can be the computer processor of the host computer, or a separate processor placed in a separate computer, are defined in a flow chart schematic to demonstrate the methodology of the real-time tracking system 300. Initially, images are acquired and imported (321) into the system. The surgical target is identified. For exemplary purposes, and not limitation, the surgical target is a breast lesion to be biopsied. Data is acquired (324) from IMU sensors which are attached to a surgical instrument. The sensor information is converted into position using a computer algorithm (325). Then, using the pre-acquired images at 321 and the computed algorithm to define position, the position of the surgical instrument on the pre-acquired images is displayed in real-time (327) during the duration of the interventional procedure.

Assuming breast immobility during the biopsy procedure, the motion of the biopsy device is followed in real-time and displayed on the previously acquired images. Aspects of the invention first confirm immobilization of a patent's breast during biopsy and then obtain tracking data in the fringe field of the MRI magnet. During confirmation of breast immobility during a biopsy procedure, pixel displacement as a function of position is recorded using non-rigid image registration between a first (contrast) series and a last series in the biopsy exam. The average displacement over the breasts of four separate patients during the biopsy procedure was about 0.8 mm with higher displacements around the biopsy site, up to about 3.5 mm displacements. In another example, the 9-gauge biopsy tools have about 4 mm diameters, and larger displacements around the biopsy site are therefore expected. The low displacement, especially with the use of 9-gauge biopsy tools with diameters of about 4 mm, confirmed a rigid geometry assumption and usefulness of real-time monitoring of the surgical instrument during a biopsy procedure, while assuming that the breast anatomy remains fixed.

Thus, tracking instruments with high precision, over a relatively large region, in a strong, inhomogenous background magnetic field proves beneficial. The solution includes implementing a set of accelerometers, gyroscopes, and Hall-effect sensors to allow real-time tracking One embodiment of a real-time MRI tracking system is illustrated in the image of FIG. 5. In the system 500, in the fringe field 501 of an MRI scanner 502, an RF breast coil 504 is positioned adjacent a translation stage 506. As shown, an IMU 508 is attached to the translation stage 506 which is endowed with an optical encoder 511. For example, the translation stage enables motion in three dimensions; each of the dimensions has a “ruler” such that a laser scans the ruler as it moves. In this implementation, the translation stage and optical encoder are used to validate the position determination reported by the IMU in comparison with the position determination reported by the optical encoder. It is to be understood, however, that this was done for validation purposes. In the clinical implementation, once the precision of the IMU of reporting position is confirmed (e.g., during the development stage), the translation stage and the optical encoder are removed. In embodiments of the invention, the IMU sensors are attached to the clinical instrument using a snap-on box, for example. Any number of attachment mechanisms may be implemented including adhering the IMU to an internal or external side wall of the instrument, implementing a pre-molded box (e.g. injection molding with the instrument) during manufacture to position the IMU, or any other method as known in the art to integrate the IMU with the instrument. In one aspect, the translation stage is used for accuracy in providing a reference from the optical encoder.

Multiple Sensor Approach

A simulated magnetic field map of a 3T MR scanner indicates background fringe fields of 100-300G, and field gradients of 4-7 G/cm (depending on the axis) in the general area where breast biopsies are performed. The spatially varying features of the field are used to establish a correspondence between position and magnetic field measurements. Unfortunately, fringe field measurements alone cannot fully determine position; thus, the orientation of the sensors (e.g., direction cosine matrix) is utilized to relate the instrument's frame of reference back to the laboratory frame where the map of the magnetic field exists.

To track position and pose of the instrument with high accuracy and at a high update rate, a sensor combination of gyroscopes, accelerometers and magnetometers, an inertial measurement unit (IMU), is installed at the distal end of an instrument. (See FIG. 3. IMU 305 is attached to surgical instrument 306.) The instrument may be any surgical instrument, catheter, probe, or instrument for medical or other purposes, such use and capability defined within a field of use. The IMUs are small MEMS devices, magnetic field compatible, and are used for different purposes in the MRI environment. For exemplary purposes, sub-degree precision has recently been shown for attitude tracking control of a handheld instrument using an IMU, and now further encompasses position tracking as described herein.

The algorithmic approach for optimal fusion of sensor measurements and use of the pre-mapped magnetic field is based on probabilistic techniques. Such techniques were applied with great success to similar problems, such as human motion tracking, indoor localization of wireless devices, and mobile robot navigation. For this problem, an occupancy grid map of the environment is correlated with measurements obtained from a laser range finder. In one case, a set of magnetic sensors are analogous to the laser range finder, since the measurements are directly correlated to position. The magnetic field map is analogous to the occupancy grid map. The goal of the algorithm is to estimate the state of the system comprising: position, velocity, acceleration, and angular velocities with respect to the laboratory frame. To model drift and bias in some elements of the IMU, the state vector may be extended in other inertial navigation applications.

The basis for the solution comprises in a two-step recursive algorithm, known as Bayes' filter. In Bayes' general form, the filtering process has two main steps, prediction and update. For prediction, the probability density function associated with the state at iteration k, a.k.a. belief (b̅e̅l̅(xₖ)), is estimated from the previous estimate (bel(xₖ₋₁)), using b̅e̅l̅(xₖ)=ƒₖ₋₁(bel(xₖ₋₁)). In this probabilistic framework, the state and its associated uncertainty are propagated through the non-linear function ƒₖ₋₁ derived from the kinematics associated with the sensor configuration. For the update step, the probability of obtaining measurements zₖ given the state xₖ, p(zₖ|xₖ), is used to correct the prediction generated in previous step (b̅e̅l̅(xₖ)), through bel(xₖ)=ηp(zₖ|xₖ)b̅e̅l̅(xₖ). To update the predicted state, 1) the magnetic map, which relates magnetometer measurements with the instrument's position and 2) the inertial measurements, which relate to the instrument's orientation and motion are considered. Furthermore, the probabilistic nature of p(zₖ|xₖ) allows for incorporating measurement noise and map errors, in a similar way range finder and occupancy grid map errors are accounted for in mobile robot navigation.

Considering the non-linear relationships of the application, a sampled representation of belief (particle filter) enables assessment of the performance bounds of the algorithm and error budgets. To reduce computational demand, parametric representations are evaluated, such as unscented Kalman filtering. Generic implementations of these algorithms in optimal estimation libraries have been developed for this application, as well as multiple others.

While the use of a simulated fringe field map for positioning could be utilized, for greater accuracy the fringe fields are mapped in the biopsy region using an automated, MR compatible translation stage on MRI scanners, and compared to the simulated field maps. If the simulated field enables accurate sensor localization, this map is preserved as the standard; otherwise, the measured maps are set as a reference. In the latter case, a limited set of (corner) measurements may be performed and used to interpolate the fields.

In addition, the stability of measurements of the sensors attached to the surgical instrument may be affected by the mechanical instabilities created in the MRI room (such as the motion of nearby elevators). The sensors enable correction through field referencing. If the measurements are sensitive to disturbances, other sensors may be added to the compression grid, for example, to enable correction for such effects.

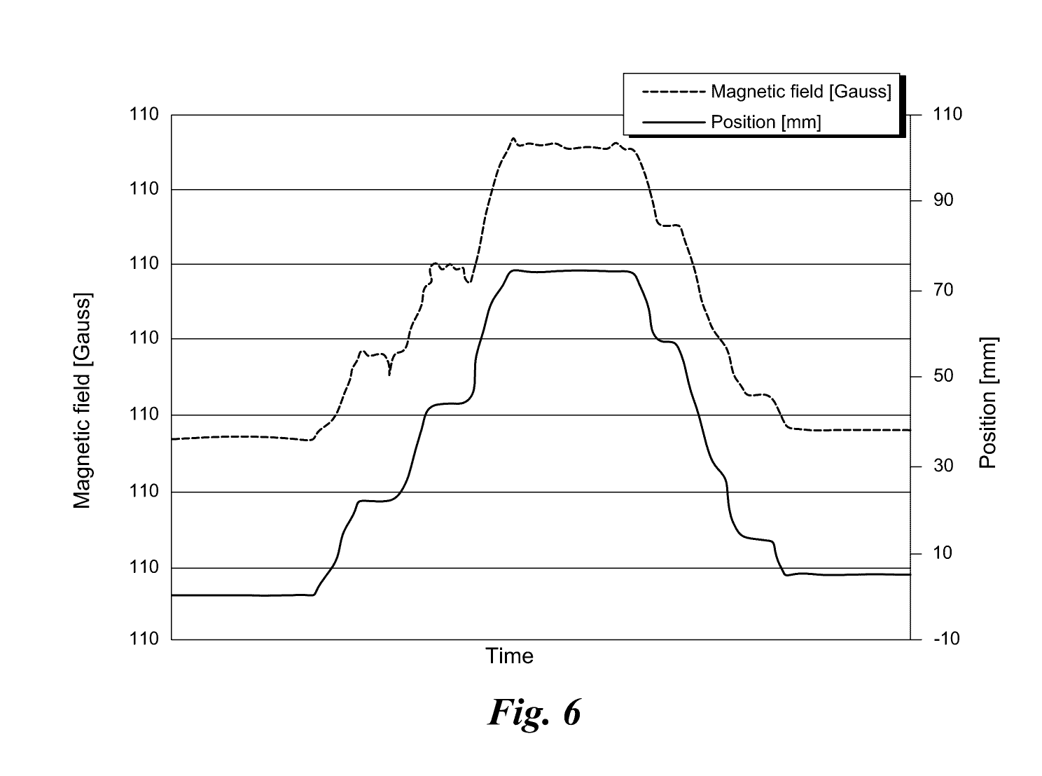

One embodiment, as shown in FIG. 6, displays the magnetic field reported by the IMU magnetometer [sensors] 508 and the position reported by the optical encoder 511 of the translation stage 506 as a function of time, while moving the translation stage over about 8 cm. This confirms a direct relationship between the field reported by the sensor and position; this graph indicates that measurements of millimeter precision are achievable. Once a map of the background field in the biopsy space is uploaded in the biopsy workstation, the magnetic field measurement translates to position.

In order for the advancement of the biopsy tool to be displayed on the previously acquired MRI images, a common reference frame needs to be established for the lesion and the biopsy tool. The lesion is visualized on the MRI images. These images are displayed in the patient reference frame, which depends on the landmark location. The biopsy tool is visualized in the laboratory frame, which can have identical orientation (angles) as the patient reference frame, but is offset in all three directions versus the patient frame. Fiducial(s) embedded in the compression grid 314, visible in the MRI images and accessible during real time biopsy instrument tracking, enable superposition of data acquired in these two (2) frames of reference. An initial calibration step, (e.g., the contact between the tip of the tracked biopsy tool and these fiducials) determines the transformation matrix that links the two reference frames. The fiducials can have the form of liquid-filled vials. Correction for susceptibility induced magnetic field changes may be implemented in order to increase the accuracy of localizing the fiducial versus the lesion.

In another aspect, an optical tracking or RFID based tracking may be utilized. Optical tracking, however, is difficult in this situation, as the radiologist's hand can come between the instrument and the source of light/detector. RFID based tracking also uses a transmitter, and is usually not very accurate.

The invention disclosed herein provides a solution to resolve issues around performing a biopsy blindly. As a biopsy device advances toward a lesion in real time, the biopsy device can now be visualized in relation to the location of the lesion, and tracked in real-time. This methodology not only enhances accuracy but also shortens the procedure time.

The various embodiments may be implemented in connection with different types of systems including a single modality imaging system and/or the various embodiments may be implemented in or with multi-modality imaging systems. The system is illustrated as an MRI imaging system and may be combined with different types of medical imaging systems, such as a Computed Tomography (CT), Positron Emission Tomography (PET), a Single Photon Emission Computed Tomography (SPECT), as well as an ultrasound system, or any other system capable of generating images, particularly of a human. Moreover, the various embodiments are not limited to medical imaging systems for imaging human subjects, but may include veterinary or non-medical systems for imaging animals and primates.

It should be noted that the particular arrangement of components (e.g., the number, types, placement, or the like) of the illustrated embodiments may be modified in various embodiments. Different numbers of a given module or unit may be employed, a different type or types of a given module or unit may be utilized, a number of modules or units (or aspects thereof) may be combined, a given module or unit may be divided into plural modules (or sub-modules) or units (or sub-units), a given module or unit may be added, or a given module or unit may be omitted.

It should be noted that the various embodiments may be implemented in hardware, software or a combination thereof. The various embodiments and/or components, for example, the modules, or components and controllers therein, also may be implemented as part of one or more computers or processors. The computer or processor may include a computing device, an input device, a display unit and an interface, for example, for accessing the Internet. The computer or processor may include a microprocessor. The microprocessor may be connected to a communication bus. The computer or processor may also include a memory. The memory may include Random Access Memory (RAM) and Read Only Memory (ROM). The computer or processor further may include a storage device, which may be a hard disk drive or a removable storage drive such as a solid state drive, optical drive, and the like. The storage device may also be other similar means for loading computer programs or other instructions into the computer or processor. Use of a robot in the magnet and/or to perform the biopsy under MR imaging guidance may also be implemented. In other embodiments, various tissues in other parts of the human or animal body can be imaged.

As used herein, the term “computer,” “controller,” and “module” may each include any processor-based or microprocessor-based system including systems using microcontrollers, reduced instruction set computers (RISC), application specific integrated circuits (ASICs), logic circuits, GPUs, FPGAs, and any other circuit or processor capable of executing the functions described herein. The above examples are exemplary only, and are thus not intended to limit in any way the definition and/or meaning of the term “module” or “computer.”

The computer, module, or processor executes a set of instructions that are stored in one or more storage elements, in order to process input data. The storage elements may also store data or other information as desired or needed. The storage element may be in the form of an information source or a physical memory element within a processing machine.

The set of instructions may include various commands that instruct the computer, module, or processor as a processing machine to perform specific operations such as the methods and processes of the various embodiments described and/or illustrated herein. The set of instructions may be in the form of a software program. The software may be in various forms such as system software or application software and which may be embodied as a tangible and non-transitory computer readable medium. Further, the software may be in the form of a collection of separate programs or modules, a program module within a larger program or a portion of a program module. The software also may include modular programming in the form of object-oriented programming. The processing of input data by the processing machine may be in response to operator commands, or in response to results of previous processing, or in response to a request made by another processing machine.

As used herein, the terms “software” and “firmware” are interchangeable, and include any computer program stored in memory for execution by a computer, including RAM memory, ROM memory, EPROM memory, EEPROM memory, and non-volatile RAM (NVRAM) memory. The above memory types are exemplary only, and are thus not limiting as to the types of memory usable for storage of a computer program. The individual components of the various embodiments may be virtualized and hosted by a cloud type computational environment, for example to allow for dynamic allocation of computational power, without requiring the user concerning the location, configuration, and/or specific hardware of the computer system.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the invention without departing from its scope. Dimensions, types of materials, orientations of the various components, and the number and positions of the various components described herein are intended to define parameters of certain embodiments, and are by no means limiting and are merely exemplary embodiments. Many other embodiments and modifications within the spirit and scope of the claims will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects.

This written description uses examples to disclose the various embodiments, and also to enable a person having ordinary skill in the art to practice the various embodiments, including making and using any devices or systems and performing any incorporated methods. The patentable scope of the various embodiments is defined by the claims, and may include other examples that occur to those skilled in the art. Such other examples are intended to be within the scope of the claims if the examples have structural elements that do not differ from the literal language of the claims, or the examples include equivalent structural elements with insubstantial differences from the literal languages of the claims.

1. A system for real-time tracking and navigation during magnetic resonance imaging (MRI) guided intervention, the system comprising:
a sensor combination attached to an instrument, wherein the sensor combination includes at least one gyroscope and at least one accelerometer, with the sensor combination recording a plurality of sensor measurements;
a computer processor executing an algorithm that relates the sensor measurements to states of the system, the states comprising position, velocity, acceleration, and angular velocity; and
a display presenting a real-time visualization of a fixed point on the instrument overlayed on a designated target;
wherein the sensor combination is an inertial measurement unit (IMU).

2. The system of claim 1, wherein the sensor combination further comprises at least one magnetometer.

3. The system of claim 1, wherein the MRI guided intervention is performed in a fringe field of an MRI scanner.

4. The system of claim 1, wherein the IMU includes at least three accelerometers, three gyroscopes, and three magnetometers along three orthogonal axes.

5. The system of claim 1, further comprising a measured or simulated three-dimensional (3D) map of a magnetic field at a location of the MRI guided intervention as used by the algorithm to increase localization accuracy.

6. The system of claim 1, wherein the fixed point on the instrument is within a tip of the instrument and is displayed virtually on the designated target, wherein the designated target is an anatomical target.

7. The system of claim 6, wherein the display depicts movement of the tip of the instrument as it advances towards the designated target during the MRI guided interventional procedure.

8. The system of claim 6, further comprising one or more images of the anatomical target acquired prior to the MRI guided intervention.

9. The system of claim 1, wherein the sensor combination is attached to a stylet or any biopsy device.

10. The system of claim 1, wherein the sensor combination includes at least one MEMS device.

11. The system of claim 1, wherein the MRI guided intervention is a biopsy.

12. The system of claim 11, wherein the biopsy is a breast biopsy.

13. The system of claim 12, wherein the IMU is located on the instrument and the breast biopsy is performed next to a breast coil in an MRI room.

14. The system of claim 1, further comprising an occupancy grid map correlated with the sensor measurements obtained from the sensor combination.

15. A system for real-time tracking and navigation during magnetic resonance imaging (MRI) guided biopsy, the system comprising:
a sensor combination attached to an instrument, the sensor combination including at least one gyroscope and at least one accelerometer, and optionally a magnetometer, wherein the sensor combination is an inertial measurement unit (IMU);
a computer processor executing an algorithm to transform the sensor measurements into states of the instrument, the states comprising position, velocity, acceleration, and angular velocity; and
a display presenting a real-time three-dimensional (3D) visualization of a tip of the instrument, wherein the display depicts positioning of the tip as it advances towards a designated target;
wherein the MR guided intervention is performed in a fringe field of an MR scanner.

16. The system of claim 15, wherein the designated target is a biopsy lesion.

17. The system of claim 15, wherein the magnetometers are Hall-effect sensors providing the components of a magnetic field in a frame of reference of the instrument, and the gyroscopes and the accelerometers provide the orientation of the instrument.

18. The method of real-time tracking and navigation during magnetic resonance imaging (MRI) guided intervention, the method comprising:
providing a system for real-time tracking and navigation during magnetic resonance imaging (MRI) guided intervention, the system comprising:
a sensor combination comprising one or more of a gyroscope, an accelerometer, and a magnetometer, the sensor combination to record a plurality of sensor measurements and attached to an instrument, wherein the sensor combination is an inertial measurement unit (IMU);
a computer processor executing an algorithm that relates the sensor measurements to states of the instrument, the states comprising position, velocity, acceleration, and angular velocity; and
a display presenting a real-time, three-dimensional (3D) visualization of a fixed point on the instrument and overlayed on a designated anatomical target;
advancing the instrument towards the designated anatomical target; and
tracking the instrument during the step of advancing, wherein the step of tracking comprises obtaining sensor measurements from the sensor combination and transforming the sensor measurements into the state of the instrument with real-time visualization.

19. The method of claim 18, wherein the state represents position, velocity, and orientation, without external reference.

20. The method of claim 19, wherein the state represents one or more of a change in the position, a change in the velocity, and a change in the orientation, alone or in combination.

21. The method of claim 18, further comprising a step of extending the algorithm to model drift and bias of the IMU.

22. The method of claim 18, further comprising a step of correcting the algorithm to update a predicted state, by incorporating noise and map errors in a probability density function, to account for occupancy grid map errors in navigation.

23. The method of claim 18, further comprising a technique of simultaneous localization and mapping (SLAM) to refine mapping of the at least one magnetic field in a biopsy region for each individual MRI scanner.

24. The method of claim 18, wherein the step of advancing comprises moving the instrument through a biopsy grid while avoiding intersection with one or more blood vessels.

25. The method of claim 18, further comprising a step of using computer-aided detection of the designated anatomical target.
